(19) United States
(12) Patent Application Publication
(10) Pub. No.: US 2025/0269070 A1
(43) Pub. Date: Aug. 28, 2025

(54) Fluorescent Marker

(71) Applicant: UCL Business PLC, London (GB)

(72) Inventors: Benjamin Michael Davis, East Hagbourne (GB); Maria Francesca Cordeiro, London (GB); Li Guo, London (GB)

(21) Appl. No.: 18/907,857

(22) Filed: Oct. 7, 2024

Related U.S. Application Data
(63) Continuation of application No. 17/050,121, filed on Oct. 23, 2020, now abandoned.

(30) Foreign Application Priority Data
Apr. 23, 2018 (GB) 1806581.3

(51) Int. Cl. A61K 49/00; A61K 9/00

(52) U.S. Cl. CPC A61K 49/0056; A61K 9/0043; A61K 49/0034; A61K 49/0043

(57) ABSTRACT

The present invention provides a method of diagnosing a CNS disorder comprising administering a fluorescent marker of retinal integrity to a subject and generating an image of the subject's eye, wherein the fluorescent marker is delivered by intranasal administration is also provided and fluorescent markers of retinal integrity for use in such methods. Also provided is a pharmaceutical composition comprising an annexin or a functional fragment or derivative thereof conjugated to a compound of 2 kDa or less, wherein the composition comprises annexin or a functional fragment or derivative thereof conjugated at a concentration of at least 5 mg/ml

### FIELD OF INVENTION

The present invention relates to fluorescent markers for use in diagnosing CNS disorders.

### BACKGROUND TO THE INVENTION

Evaluation of the retina can provide information regarding the presence and severity of many systemic diseases. For example, Detection of Apoptotic Retinal Cells (DARC) describes a technique to monitor the rate of cell death in the retina that recently completed Phase I clinical trial for the diagnosis of Glaucoma (Cordeiro et al 2017) and is presently undergoing Phase II trials in a range of neurodegenerative diseases including glaucoma, age-related macular degeneration, optic neuritis and Down's (as a model of Alzheimer's Disease). The technology currently consists of intravenously administering a novel fluorescent agent called Anx776 which comprises a modified version of the endogenous protein Annexin A5, fluorescently conjugated to a near-infrared fluorophore Dy-776 (Cordeiro et al 2017).

DARC utilizes the unique optical properties of the eye to enable the possibility of directly observing single nerve cell apoptosis in patients using a fluorescent-labelled derivative of human Annexin V. Annexin V is a human protein that has the ability to bind to phosphatidylserine (PS) in the presence of Calcium. PS is present in the plasma membrane of every cell, but apoptotic cells express PS in the outer leaflet of the plasma membrane. Annexin V binds to the exposed PS thereby identifying apoptosis.

For the phase I and phase II clinical trial of DARC Anx776 is administered intravenously. Other fluorescent molecules used for the detection of retinal disorders by confocal scanning laser ophthalmoscopy (cSLO) imaging are also predominantly administered via intravenous injection. For example, Sodium fluorescein and Indocyanine green (ICG) are commonly administered intravenously to label retinal vasculature to identify abnormal blood vessel growth (angiogenesis) and leakage associated with Age Related Macular Degeneration (AMD) (Jorzik et al 2005).

The use of intravenous administration require direct medical supervision, meaning that patients are not routinely evaluated in such a way unless or until such time as a pathology is suspected. There is therefore a need for alternative techniques that can be used to evaluate the retina in order to enable wider patient access to this type of evaluation and, potentially, earlier diagnosis of retinal and neurodegenerative diseases.

### SUMMARY OF THE INVENTION

In a first aspect, the present invention provides a fluorescent marker of retinal integrity for use in diagnosing a central nervous system (CNS) disorder, wherein the fluorescent marker is to be delivered by intranasal administration. Surprisingly, the present inventors have found that intranasal administration of fluorescent markers results in rapid accumulation of fluorescence in the retina, with retinal imaging showing results identical to those previously obtained after intravenous administration of the same fluorescent agents.

Without being bound by theory, the inventors believe that the intranasally delivered fluorescent marker of retinal integrity is systemically absorbed into the circulation. Due to the local administration the fluorescent markers of retinal integrity may require reduced doses and may have more rapid onset than systemically administered formulations of the same agents.

As used herein, a fluorescent marker of retinal integrity refers to a fluorescent marker for interrogating the health of retinal cells and/or the integrity of retinal cells and/or blood vessels. For example, the marker may identify and/or distinguish apoptotic and necrotic cells, or identify areas of blood vessel leakage or angiogenesis. The fluorescent marker emits light in response to excitation and may have an emission wavelength of about 400 nm to about 1000 nm, preferably about 500nm to about 900 nm.

The fluorescent marker of retinal integrity is preferably provided in a form suitable for topical delivery, especially intranasal delivery. The fluorescent marker of retinal integrity may be provided in the form of a pharmaceutical composition and may be in the form of a solution, a suspension or a dry powder suitable for inhalation. Pharmaceutical compositions comprising the fluorescent marker of retinal integrity may be sterile and may comprise one or more pharmaceutically acceptable carriers or excipients. Suitable carriers and excipients will be familiar to the skilled person and may be optimised in line with the intended route of intranasal delivery. For example, compositions comprising the fluorescent marker of retinal integrity may also include buffers, binders, preservatives, thickeners or antioxidants, such as trehalose.

In embodiments of the invention, the fluorescent marker of retinal integrity may be a fluorescent marker of retinal blood vessel integrity. Typically, such a fluorescent marker will enter and circulate within the retinal blood vessels, allowing abnormal blood vessel growth (angiogenesis) and/or sites of leakage to be easily visualised.

A fluorescent marker of retinal blood vessel integrity may have a molecular weight of about 2 kDa or less, or 1 kDa or less. In embodiments of the invention a fluorescent marker of retinal blood vessel integrity may have a molecular weight of about 100 Da or about 1 kDa, preferably about 300 Da to about 800 Da.

Suitable fluorescent markers of retinal blood vessel integrity for use in the present invention include fluorophores. Particularly preferred fluorescent markers of retinal blood vessel integrity include sodium fluorescein and indocyanine green (ICG).

As explained above, the fluorescent marker of retinal blood vessel integrity is to be administered intranasally and may be administered at dosages that are same or less than those given for intravenous administration of the same marker. For example, sodium fluorescein may be administered intranasally at concentrations of about 50 mg/mL to about 500 mg/mL, preferably about 50 mg/mL to about 200 mg/mL. In embodiments of the invention sodium fluorescein may be administered intranasally at about 100 mg/mL. ICG may be administered intranasally at concentrations of about 1 mg/mL to about 100 mg/mL, preferably about 25 mg/mL to about 100 mg/mL. In embodiments of the invention ICG may be administered intranasally at about 50 mg/mL.

In embodiments of the invention, the fluorescent marker of retinal integrity may be a marker of retinal cell integrity. Typically, a fluorescent marker of retinal cell integrity will comprise a fluorescent label and a marker of one or more of apoptosis, necrosis, cell activity, cell stress or protein aggregation.

Fluorescent labels refer to compounds or molecules (such as fluorophores) which emit light in response to excitation, and which may be selected for use due to increased signal-to-noise ratio and thereby improved image resolution and sensitivity while adhering to light exposure safety standard to avoid phototoxic effects. It is preferred that the fluorescent labels cause little or no inflammation on administration. The fluorescent labels may have wavelengths infrared or near-infrared ranges. The fluorescent labels may have emission wavelengths of about 400 nm to about 1000 nm, preferably about 500 nm to about 900 nm, more preferably about 700 nm to about 900 nm.

Suitable fluorescent labels include one or more of sodium fluorescein, indocyanine green (ICG), curcumin, IRDye700, IRDye800, Dy-776, Dy-488 and D-781. In preferred embodiments of the invention the fluorescent label is Dy-776.

The fluorescent marker of retinal cell integrity may be prepared using standard techniques for conjugating a fluorescent label to a marker compound. Such labels may be obtained from well-known sources such as Dyomics. Appropriate techniques for conjugating the label to the marker are known in the art and may be provided by the manufacturer of the label.

A marker of apoptosis refers to a marker that allows cells undergoing apoptosis to be distinguished from live cells. Additionally, the marker preferably should be able to distinguish apoptosing cells from necrotic cells. For example, it may be a compound or molecule that specifically binds to apoptotic cells but not to live cells or necrotic cells. Markers of apoptosis include, for example, the annexin family of proteins. Annexins are proteins that bind reversibly to cellular membranes in the presence of cations. In particular, annexins are able to bind to phosphatidylserine (PS) in the presence of calcium. PS is present in the plasma membrane of every cell, but apoptotic cells express PS in the outer leaflet of the plasma membrane. Annexins bind to the exposed PS thereby identifying apoptosis. Annexins useful in the invention may be natural or may be recombinant. The protein may be whole or maybe a functional fragment, that is to say a fragment or portion of an annexin that binds specifically to the same molecules as the whole protein. Also, functional derivatives of such proteins may be used. In particular, functional fragments or derivatives of annexins may include molecules containing an “annexin repeat”, that is a domain of approximately 70 amino acids that is conserved both within individual annexins and between members of the family. A variety of annexins are available, such as those described in US Patent Application Publication No. 2006/0134001A. A preferred annexin is Annexin 5, which is well known in the art. Other annexins that may be used include Annexins 11, 2 and 6. Other markers of apoptosis are known in the art including for example C2A domain of synaptotagmin-I, duramycin, non-peptide based isatin sulfonamide analogs, such as WC-II-89, and ApoSense, such as NST-732, DDC and ML-10 (Saint-Hubert et al., 2009).

In particularly preferred embodiments of the invention the marker of apoptosis is Annexin 128(Tait et al 2005). Annexin 128 is a variant of Annexin 5 and differs from the wild-type by two single amino acid mutations. Annexin 128 includes an exposed thiol group at the N-terminus, which affords increased conjugation efficiency for molecular tags, such as fluorescent labels.

In particularly preferred embodiments of the invention the fluorescent marker of retinal cell integrity comprises Annexin 128 conjugated to Dy-776. The Annexin 128 and Dy-776 may be conjugated at a 1:1 fluorescent label:marker ratio.

A marker of necrosis refers to a marker that allows cells undergoing necrosis to be distinguished from live cells and those undergoing apoptosis. For example it may be a compound or molecule that specifically binds to necrotic cells but not to live cells or apoptosis cells. Markers of necrosis include, for example propidium iodide (PI), an intercalating agent that binds to nucleic acid with little or no sequence preference. Other necrotic markers are known in the art including pyrophosphate, antimyosin, glucarate, hypericin and its derivatives, such as hypericin monocarboxylic acid and pamoic acid, such as bis-hydrazide-bis-DTPA pamoic acid. 99mTc-pyrophosphate, 111In-antimyosin, 99mTc-glucarate and methylene blue have been used in particular.

Other indications of cell activity can also be used to identify apoptotic or necrotic cells. For example, changes in mitochondrial function may be observed, reactive oxygen species (ROS) may be used as markers, as may calcium ions. Markers of cell activity may include one or more of membrane dyes, mitochondrial dyes, autophagy dyes, necrosis dyes and calcium flux. More particularly, markers of cell activity may include one or more of Fluo-3, N-(fluorescein-5-thiocarbamoyl)-1,2-dihexadecanoyl-sn-glycerol-3-phosphoethanolamine, JC-1, JC-9 with dual emission, reduced rhodamines and rosamines, rhodamine 123 and Di-8-ANePPS. Markers of cell stress may include one or more of a marker of lipid peroxidation, glutathione (GSH) or reactive oxygen species (ROS), such as superoxide, peroxyl radical, hydrogen peroxide, hydroxyl radical and peroxynitrite

Protein aggregation in the retina can occur intra-or extracellularly in or around neurons in the retina, but typically outside the retinal vasculature. The presence of protein aggregates is known to be correlated with cells that undergo neurodegeneration. Suitable markers of protein aggregation include one or more of congo-red, curcumin or Thioflavin S.

As described above, the present invention provides an intranasally delivered fluorescent marker of retinal integrity for use in diagnosing a CNS disorder in a subject. The subject is preferably a mammal, including a human, and may be a paediatric or geriatric patient. It is possible to identify a CNS disorder by analysing retinal integrity (see e.g. methods described in WO 2009/077750 and WO 2011/055121). For example, the fluorescent marker of retinal integrity can be used to demonstrate the distribution of cell death in the retina by labelling apoptotic and/or necrotic cells. From that distribution it is possible to differentiate between different neurodegenerative diseases which have distinct patterns of apoptotic and/or necrotic activity.

The CNS disorder may be inflammatory (such as arthridides or granulomatous), infective (such as viral, encephalitic, or bacterial), vascular (such as angiogenic, occlusive or metabolic), or degenerative (such as glaucoma, age-related macular degeneration (AMD), Alzheimer's disease, or Parkinson's disease). In embodiments of the invention the CNS disorder may be a neurodegenerative disease, especially an ocular neurodegenerative disease. The term “ocular neurodegenerative diseases” is well-known to those skilled in the art and refers to diseases caused by gradual and progressive loss of ocular neurons. They include, but are not limited to glaucoma, AMD, optic neuritis and diabetic retinopathy. Neurodegenerative diseases include, for example, Parkinson's disease, Alzheimer's disease, Huntington's disease and Friedreich's ataxia. In embodiments of the invention the CNS disorder may include traumatic brain injury, stroke, cerebral palsy, e.g. as caused by neonatal hypoxia, and cancer, including brain tumours.

The present invention additionally provides a method for diagnosing a CNS disorder, the method comprising administering a fluorescent marker of retinal integrity as described herein to patient and generating an image of the patient's retina, wherein the fluorescent marker of retinal integrity is administered intranasally. The image of the patient's retina is preferably generated in vivo and may be obtained using cSLO imaging. The presence or absence of a fluorescent signal in the image may indicate the presence or absence of a CNS disorder, thereby enabling a clinician to diagnose the presence or absence of disease. If a fluorescent signal is present in the image the location and distribution of the fluorescence can allow the clinician to differentiate types of CNS disorder. For example, the fluorescent signal my indicate angiogenesis or sites of blood vessel leakage. Alternatively, the fluorescent signal may indicate the number and/or pattern of distribution of apoptotic and/or necrotic cells.

The diagnostic method can additionally be used to monitor disease progression, for example, to evaluate the effectiveness of a treatment or stage the disease. A first in vivo image of a patient's eye can be compared with a later in vivo image of the same patient's eye that is taken days, weeks or months after the first image. Changes in the fluorescent signal can indicate disease progression or an effective treatment. For example, an increase in the fluorescent signal may indicate an increase in the number of apoptosing cells, which likely indicates disease progression. Images may be analysed as described in WO 2011/055121.

In a further aspect the present invention provides a pharmaceutical composition comprising a marker of retinal cell integrity as described above, wherein the composition comprises an annexin or a functional fragment or derivative thereof conjugated to a compound of 2 kDa or less, wherein the annexin or functional fragment or derivative thereof is present at a concentration of at least 5 mg/ml. Compositions comprising annexins are known to encounter problems with precipitation when the annexins are present at concentrations of around 2 mg/ml or more. However, the present inventors have been able to solve this problem in order to provide compositions containing an annexin at a concentration of at least 5 mg/ml. The compositions have demonstrated stable storage for up to 50 days at 25° C. when protected from light. Without being bound by theory, the present inventors believe that conjugation of a compound of 2 kDa or less to the N-terminus of the annexin protein acts to sterically impede protein aggregation.

The conjugation may be electrostatic or covalent. The compound is preferably conjugated to the N-terminus (i.e. the first 30 amino acids) of the annexin or functional fragment or derivative thereof. In embodiments of the invention the annexin or functional fragment or derivative thereof may be present at a concentration of about 5 mg/ml to about 20 mg/ml, preferably about 5 mg/ml to about 10 mg/ml.

The compound may be an organic compound or an inorganic compound, and may have a size of from about 100 Da to about 2 kDa, preferably about 200 Da to about 1 kDa, more preferably about 300 Da to about 800 Da. The compound may be a fluorescent label as described above, or may be a therapeutic agent. Suitable fluorescent labels may be selected from one or more of sodium fluorescein, indocyanine green (ICG), curcumin, IRDye700, IRDye800, Dy-776, Dy-488 and D-781. In preferred embodiments of the invention the fluorescent label is Dy-776.

In preferred embodiments of the invention the pharmaceutical composition comprises annexin 128 conjugated to Dy776. Surprisingly, several methods for concentrating the marker of retinal cell integrity have been assessed and have demonstrated that annexin 128 conjugated to Dy776 is more stable at high concentrations than annexin 5.

The pharmaceutical composition is preferably in a form suitable for intranasal administration (such as a solution, a suspension or a dry powder suitable for inhalation), and may comprise suitable carriers and/or excipients as discussed above. The pharmaceutical composition may be in the form of lyophilised powder and may be administered to a patient as a dry powder suitable for inhalation. Alternatively, the lyophilised powered may be rehydrated to form a suspension prior to administration.

### BRIEF DESCRIPTION OF THE DRAWINGS

The invention will now be described in detail, by way of example only, with reference to the figures.

FIG. 1. Intranasal administration of Anx776 rapidly reaches the retina in mice and labels apoptotic retinal cells. cSLO images of the same C57BL/6J mice eyes at baseline [A,B], and 3 hours after intranasal administration of Anx776 [C, D] and intravitreal 4% DMSO [B,D]. Note the appearance of apoptosis (white spots) is seen only in the eye given intravitreal 4% DMSO [D]. [E] Collated DARC spots at baseline and 3 h after IN Anx776 administration. [F] A Phosphatidylserine binding assay demonstrating that lyophilised concentrated Anx776 retains good PS binding activity compared to fresh Anx776 material after storage for 50 days at 25° C. while protecting from light (EC50 ratio<2).

FIG. 2. cSLO images after administration of 0.025 mL Sodium fluorescein (20% w/v) in C57BL/6J mice (488 nm excitation).

FIG. 3. Intranasal administration of 0.025 mL Indocyanine Green (ICG) in C57BL/6J mice (795 nm excitation).

### EXAMPLES

### a) Anx776 Rapidly Reaches the Circulation and Retina After Intranasal Administration

Annexin 128 was conjugated to Dy-776 in its maleimide form, providing a marker of retinal cell integrity identified as Anx776. The formulation of Anx776 presently used in the clinic (0.2 mg/mL) was concentrated 25 times to 5 mg/mL using a 5 kDa MWCO filter, within a buffer containing 20 mM Sodium Citrate, 280 mM Dextrose, pH 6.2 in water for injection.

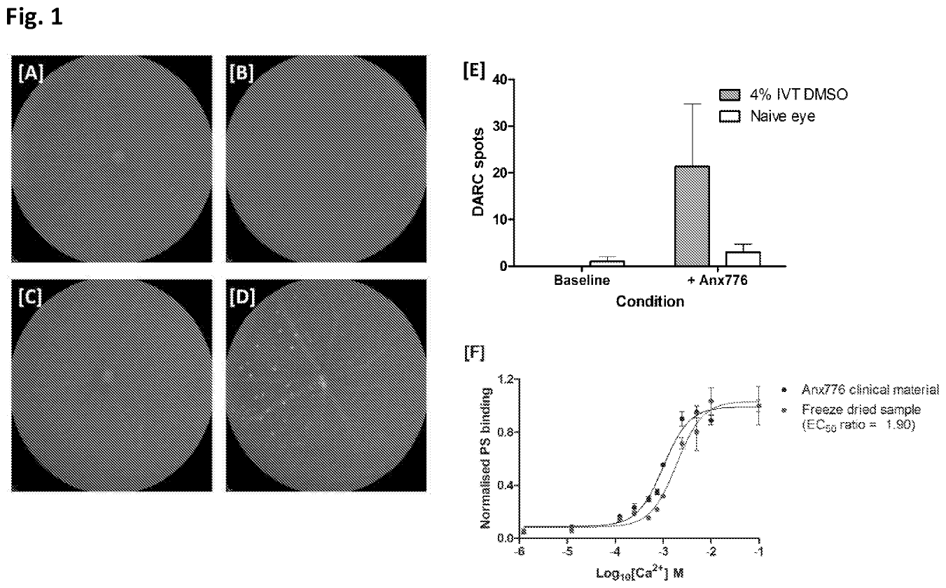

Concentrated Anx776 (hiAnx776) was tested in vivo. hiAnx776 was administered intranasally (25 μL) to C57BL/6J mice at the same time as intravitreal 4% DMSO (1 μL injection volume, PBS buffer) was injected to induce retinal cell apoptosis, using a well-established model [6]. FIG. 1 [A-B] illustrate representative cSLO images of naïve (FIG. 1A) and DMSO-insulted (FIG. 1B) retina at baseline, before treatment. These same eyes are imaged 3 hours after treatment with 4% DMSO injection (FIG. 1D) and intranasal Anx776 (FIG. 1C and FIG. 1D). Bright DARC spots are clearly visible in the DMSO-treated eyes (FIG. 1D) providing the first evidence to suggest that intranasal administration of Anx776 can reach the retina at sufficient concentrations to label apoptotic retinal cells in a similar manner to intravenously or intravitreally administered DARC. Manual quantification of DARC spots indicates an elevation in apoptosis in eyes subjected to intravitreal DMSO insults, in agreement with previous observations with intravitreally administered DARC in this model (Jorzik et al 2005).

We hypothesize Anx776 is systemically absorbed after inhalation into the circulation, and gains entry to the retina. Our experimental and clinical data with Anx776 confirms that systemic Anx776 can reach the retina after intravenous administration (Cordeiro et al 2017).

Anx776 can be readily formulated as a lyophilised powder (with Trehalose as a cryoprotectant) which retains PS binding activity for up to 50 days after storage at 25C (FIG. 1F), protecting from light. Anx776 powders containing up to 10 mg/mL of Anx776 have been prepared which could be rehydrated prior to IN delivery or alternatively administered directly as a powder formulation.

b) Small fluorescent molecules presently used for the diagnosis of retinal disorders rapidly reach the circulation in sufficient quantities to be of diagnostic use after intranasal application

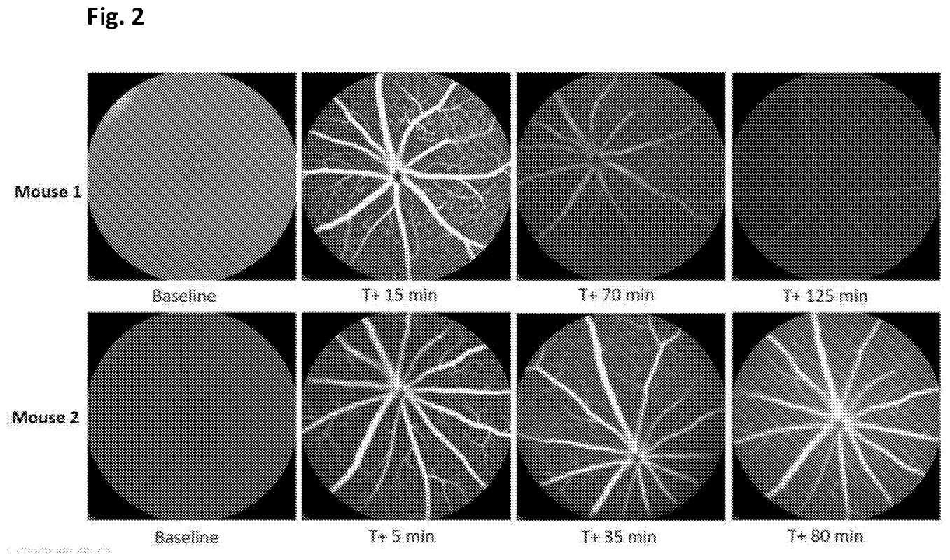

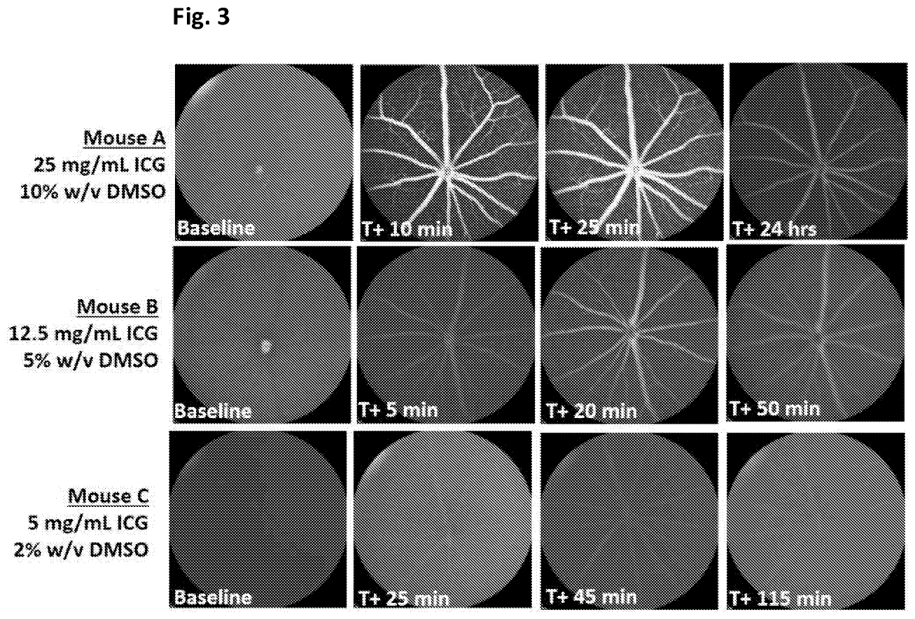

Having observed that the fluorescent protein Anx776 (36 kDa) enters the circulation upon intranasal administration, we next sought to determine whether small fluorescent molecules (Sodium fluorescein and ICG, 376 Da and 775 Da respectively) presently administered intravenously for the diagnosis of retinal disorders could also be delivered in this manner. Intranasal administration of Sodium fluorescein (FIG. 2) or ICG (FIG. 3) were both found to result in rapid accumulation of fluorescence in retinal vasculature and choroid. The resulting images acquired by cSLO were identical to those previously obtained after intravenous administration of the same fluorescent agents (Kumar et al 2014) and due to the localised administration, may require reduced doses and have a more rapid onset than systemically administered formulations of these agents.

### REFERENCES

M. F. Cordeiro, E. M. Normando, M. J. Cardoso, S. Miodragovic, S. Jeylani, B. M. Davis, L. Guo, S. Ourselin, R. A′Hern, P. A. Bloom, Brain 2017, 274, 61.
J. J. Jorzik, A. Bindewald, S. Dithmar, F. G. Holz, Retina 2005, 25, 405.
S. Kumar, Z. Berriochoa, A. D. Jones, Y. Fu, J. Vis. Exp. 2014, e51061.
De Saint-Hubert M, Prinsen K, Mortelmans L, Verbruggen A, Mottaghy FM. Methods. 2009 Jun;48 (2):178-87

JF. Tait, C. Smith, FG. Blankenberg. J Nucl Med May 1, 2005 vol. 46 no. 5 807-815

What is claimed is:

1-16. (canceled)

17. A method of diagnosing a CNS disorder comprising administering a fluorescent marker of retinal integrity to a subject and generating an image of the subject's eye, wherein the fluorescent marker is delivered by intranasal administration.

18. The method according to claim 17, wherein the fluorescent marker is in the form of a powder, a suspension or a solution.

19. The method according to claim 17, wherein the fluorescent marker is a marker of retinal blood vessel integrity.

20. The method according to claim 17, wherein the fluorescent marker has a molecular weight of about 2 kDa or less.

21. The method according to claim 17, wherein the fluorescent marker is selected from one or more of sodium fluorescein or indocyanine green (ICG).

22. The method according to claim 17, wherein the fluorescent marker is a marker of retinal cell integrity.

23. The method according to claim 17, wherein the fluorescent marker comprises a fluorescent label and a marker of one or more of apoptosis, necrosis, cell activity, cell stress or protein aggregation.

24. The method according to claim 23, wherein the fluorescent label has an emission wavelength of about 400 nm to about 1000 nm.

25. The method according to claim 23, wherein the fluorescent label is selected from one or more of sodium fluorescein, indocyanine green (ICG), curcumin, IRDye700, IRDye800, Dy-776, Dy-488 and D-781.

26. The method according to claim 23, wherein the marker of apoptosis is selected from one or more of an annexin, C2A domain of synaptotagmin-I, duramycin, non-peptide based isatin sulfonamide analogs, such as WC-II-89, and ApoSense, such as NST-732, DDC and ML-10.

27. The method according to claim 26, wherein the annexin is selected from one or more of annexin 2, annexin 5, annexin 6, annexin 11 or annexin 128.

28. The method according to claim 23, wherein the marker of necrosis is selected from one or more of propidium iodide (PI), pyrophosphate, antimyosin, glucarate, hypericin, hypericin monocarboxylic acid, pamoic acid, bis-hydrazide-bis-DTPA pamoic acid, 99mTc-pyrophosphate, 111 In-antimyosin, 99mTc-glucarate and methylene blue.

29. The method according to claim 23, wherein the marker of cell activity is selected from one or more of membrane dyes, mitochondrial dyes, autophagy dyes, necrosis dyes and calcium flux.

30. The method according to claim 23, wherein the marker of cell stress is selected from one or more of a marker of lipid peroxidation, glutathione (GSH) or reactive oxygen species (ROS), such as superoxide, peroxyl radical, hydrogen peroxide, hydroxyl radical and peroxynitrite.

31. The method according to claim 23, wherein the marker of protein aggregation is selected from one or more of congo-red, curcumin or Thioflavin S.

32. The method according to claim 17, wherein the CNS disorder is inflammatory (such as arthridides or granulomatous), infective (such as viral, encephalitic, or bacterial), vascular (such as angiogenic, occlusive or metabolic), or degenerative (such as glaucoma, age-related macular degeneration (AMD), Alzheimer's disease, or Parkinson's disease).

33. A pharmaceutical composition comprising an annexin or a functional fragment or derivative thereof conjugated to a compound of 2 kDa or less, wherein the composition comprises annexin or a functional fragment or derivative thereof conjugated at a concentration of at least 5 mg/ml.

34. The pharmaceutical composition according to claim 33, wherein the annexin or functional fragment or derivative thereof is annexin 128 and wherein the compound of 2 kDa or less is Dy776.
